SEQUENCE LISTING (1 sequence)

SEQ ID NO: 1 - DNA, 8354 bp, Mycoplasma gallisepticum
agaccaaacttccctaaccaaatgcctaatatgaaccaacctagaccaggtttcagaccacaacctggtggtggggtgccgatgggaaataaagccggaggtgggtttaatcacccaggtacaccaatgggtccaaaccgcacgaacttccctaatcaaggaatgaatcagcccccacacatggcaggaccaagagctggttttccaccacaaaatggacctagataagactttagaaaactaaaacttaatctttatcaactaaaaaaaaatatttaaaccaaaaatattattattaaatcttttaaactttttatatatttatataataataattaaatctttaaataacctaatgtttgttaacatagtagaagaatttaaatcgtgaaaaaacttatttttaaattatcagtcggaataactcctcttgccttaatcggtttaggtagttttggattagcagtttctggagctaagccaaataaccttaaacctgttaaccaagttggggaaatgaattcacaaggccaatctaatcttttagagaaagctcgtagatgaagaaactctaacttcacatcactttcaattgacggtaccaacccaggtgcattagttttaactggatcaaaatcaatthgccggattgatttgtatggtaacgtgatttgaacgtttgatccaggtaacacaaacgatctaactggtaaggttggattttatgatgctaacaatagattgactgcattttctggagacgttccttttaatgtaagtgatctaagctctaaaacagttgtagaagctactcaagatcaagaagatcctaatgttttctacttattattaattccagatgcagcggttcaacaagaacaaaagactaaagatcaagtgtttgaaaactactttatgtctgatgcacctgctactggtgatactagcgctgaaggttctgcaactcctgctggtggtggttcaagtagtagtgctgctggaggaggtgctgttgctcctgctgctgcgagttcgactgctagacttgttgaagaagggaatagtgctggtatgggaacgatgactcctactgcttctacttctgaaacagttatagattataatagcgatcaaaataaaattcctaaacctaaaacactattagacagtagcgaaagttctgaaagtatcaatggtggaagaacatatgcgaacattaacactcagaataatttacaaggtgttattgtaaaagttaacgaaaatttatttaattcggaaaatccctttgcagtagaaaatatggcgttcattaagccgaaggatatggttgataattatccttctacttgaacacaaggttctgctaacggtaaaatgactaacgttcttcaatgctacaaacatgataatcctaatgctgttaacaatagattctatagagcaaaatactatcctaaacgtttagaaactcaaacaactactcctctaattgatagttctttctctccatatgagcatccagaatgatatgaagataatcaatttgtaatgccgtgaatgcagtacataacaaatttaggtggtttatatgctaaagatggaatggtgtacctatttggtggtaacggtacgtgagttaacaacgaaagtgcattaagtattggtgttttcagaactaaatttgaaaacagaactgctgaagctccaggaaacactaaaactgttggttatccatacggtattttattatcagcgatttcttttgatgctactagaaatggattagcgcttgctcctgcaagtcttggtcaagatgttggttatcactttgttcctcgtcttgcagtgggtggtgtaagttcacctagaggagctaacggtaatattttcttaggttcagctattacttgaggaacaaacggtggtaatttcttagatactaaatgacacagtcctgctgtcattgaagatgcacctactacttttgtgactgttaatagtagtggtgcgcttcagaatagtggaaatccacaaccaacttctactccgatgcctaatagtaacggtaatgaaagcatcccttatagatgaacgaattcttatgattacaactctgtaagatttgcagctctaattagtaagccagctggtggaaacacaaaacaagttgaatcattatttacaaccgctttaaaattagatacattaaattctttaccaaataaatttactcaagaaaataatatcttctttagttatgctatgttagatggtcgtcaatgaagtttaggtacacgaaaagacagcgcatgattagcaactaatactattaataacttcacttataatactcaacaacaattagcgtctacagtagctggagaaaacgctaatccaagaaatatcttaaacgctttaacaactgcaaaagggtttgatcgaagagatattggtaatgtagtatatacttattctaataatactaataagtttacttattactatcaagttggtggcgcgattacaacttggccagaagttcaagtaaattacaaaacttcggctaatattacttactacaatttaactagaactgattttggaagtactactcctgcaactcaagatgcaaataccgtatcatctaaattaaacggcgcttacttatcatcaactggcgatcaacaaggatgatacaacggttcaatctatgttaaaaaagcgagctttacaccaagtagccaaggttatacttgacaagatttcaaaggtttaacaactacagcaagtaacgcagttatttctaactgaacaaaagctggatacagtattagaccagatgatgatacagtattcaacgcttctaagattccttttgaaaaagaaatcaccgctgctgttaatgtaagatcattagatagttactatgtacaattaaatggtgaaacttcagttaatactgtagctagagtaagtcctgattctagcgctttagccctaaaccctaacagaattactaacccattgatgaatagagataacgtaatcggtcaaggtgcgttcattagtagaaatgatattccatcatcattctttgaaaacaaaattaatgatattgtaactacagaagctgatggtaaagaagtattagatagtaaatacattaattcgatctatagatatactccacctcaaaacaatcctgatattagattaagattattagtaattgatcgttctagagcaaccaatgacttcattaagttattacctcaagtattagttgatggcgaatacgttgctgttccacaagctaatagtgtgtttgtgtctgaccaagaatttactggttttgatgcgcttccaggttatgtattaccagtagcgatctcgattccgatcatcataattgccttggcattagctttaggtctaggtattggtattccaatgtctcaaaaccgtaagatgttgaaacaaggatttgcgatttcaaacaaaaaagttgatattctgacaacagccgttggtagtgtgttcaaacaaattattaatcgaacatctgtgacaaatattaagaagacyccacaaatgcttcaagccaacaagaaagatggagcatcttcaccaagcaagccatcagctccagctgctaagaaaccagcaggaccaactaaaccatctgctccaggggcaaaaccaacagcaccagctaaaccaaaagctccagcaccaactaagaaaattgaataattaaggtaatatattaaagatatgaatatttctaaaaaacttaaaagttatacattgataggtggattagctgtatttggagctcttggttctgcaagctttggctttaagcaatcagataagagtaacgataacacgcaattagttaatcaagcaagaacgctagatgctaattctgttagacttgcaggtcttggacaaaatggttcgttgttcaatacagttcttagagatgttgatgataactttataacagcagctaatggaacaattatcaaattagatagttttactaaaccattatatggtttagatctaagtgatgattttgctggatacaaagtaaaacaaatagtttcagattacacaactagcagaaatagatttgatcaaagacaaacaagagcatattatgctctgttggttaatgatgaagctaacgttcatttaaaaagaattaatactaactcaaatagaattggtaatagaaacaacaattctaagtttgtaattggtggtgttgataatccagctcacgtaattagatttactgatgatgggactaaatttaattttacaaagcaaactcaaggtgaaattgttaatgacttcattttagatgcgccaatcttacctaaagatttacacccagattgatataacttatacattcaaagaaagatcttaccaaatgacgtaaacactgcagttgttccttgaccagtaggtagagttagtggaacaaatgctgatgatgggatgtttgattttgggaatggtcaaataactaatacagatcctattgctcaaactaaaaccactactgataatcaaaatccttcaacttttaattcaggagcaatgcctggtgcaaacaatagatacgattctcaattgaatgtcaagcatagaattaaaacatctttccaattagatgaaaaatttgtttatccagaatgaactggttctgaagagaataaaaatattacaagattagctactggaagtttgccaagcaacgaaagatattgaattcttgacataccagggactccacaagttactttaaaagaagattcagttaacgtattttcaagactatacttaaactcagttaattctttatcattcattggtgatagtatttatatttttggtacttctgaattaccatcattatgatactattcattcccaactagattatctgatctaaccgctttgaatcaagttaaaacagatgatattgaagcttcaagcactgataacggtacaacaacaaacggaacaacgacaacaactgatacatctagtggttcaacaggtgctggaacaggaaatactactaacacttctcaaacagtttctaatcctactttaaatacttatcgtagttttggaattgatagtaaaccaacttctgcaaacaaaatagatgaaactaattgagcagatcctaatgttattgaagcaagaatatatgctgaatacagattaggtattcaaaatgaaattccaataactaatgcaggaaactttatccgaaacacaattggtggtgttggttttacttcaacaggttcaagagtagttttaagagcttcttataacggtgatcaacgtccaactggaaacttccaacctttcttatacgtatttggttatttaggataccaacaaactagaacaggaactttctgatacggaacatataaactattaaacaacagcccttacgacgtattagatgctgcaagagtaggtactgaaaccaatcaatttagaagaacttcattaacataccctgttatgggtggatayctaactgaagaaggtgctagaagtttctctaatactccatatataagagcacaaggtgacacaccagaaagccgaagcatcttccaatctggctaytctgataatacttatgagtacattcaatcagttttaggatttgatggaattagaaataacttaaatgttggggttaaagcatcaagcttcttaaactcaaatagaccaaatccaaacggtctagaaatgattgctgcaacaacatacttaagatcacaaattggattagctagaacatctggattaccaaaccaacaaccattcggaacaactcaccaagttatttcagtatcacctggtgatcagttctcatcaattaagaatattagaacaatcttccctggtaaccagttatgatacttcttattcacaaatgaaaataataaatctagtgtttatacattaagattagctgactcaagtaaccctgatgcgtcaagctcattcagtccaacaagtttaattgacgttaatgaaattggtgtaatcttacctttattagacaattcattctatacagtaaatgctgctggtaatgttgcattgttctcatcaaaccctggttctcctggatcatatactgctgtaaatacatttaatcagaacttatctgatattgcttttgaaggttctggtgctaaatatacatctgatttctgaggaacaatccaattcaaacccgatgagtacttaattcaaaatgggttcactagtcaagtggctagaaacttcgttacaaaccaaagcttcttaaacagtttagttgacttcactcctgctaatgctggtactaactaccgtgtagtggttgatcctgatggtaatttaacaaaccaaaacctacctctaaaagttcagatccaatacttagatggtaagtattatgatgctaaattaaagaacaataatttagtaacattctcttataacaactttgctgctttaccttcatgagtagtgcctacagcaattggtagtacattaggtattcttgcaattatgatcatcttaggattagctatcggtattcctttaagagctcaaagaaaattacaagacaaagggttcaaaacaacattcaaaaaagttgataccttgactgctgctgttggttcagtttacaagaagattattacccaaactgctaacgttaagaaaaaacctgctgctttaggtgctggtaaatctggtgataagaaacctgctgctgctgctaaacctgctgctccagctaaaccatctgcaccaaaagctagctcaccagctaaaccaactgcgcctaaatctggtgcgcctacaaaaccaactgctcctaagccagctgctccaaaaccaaccgctcccaaagaataaaaataaaacttcttagttttaaaaaataacctcccttctctacgataaggtaatcattacctctatcaaaaaagggtggttgttttatgtataataattacatactaaatttagttagattaaccaagtaaataatcaatttagttatcatcaaaaagataatgttaaaagctcacatcaaaagatcgcttattctttttattatcttactaatagctgcgattagtttaatttttatcggtttgtttggttttaaaaaaccaaacattaatcaagttaaactaaacaaccaatcaagtagtttaattaccaaccaaccctctgaccagatcattagaaaatattctttagattcgattagatgaaatgctaatgctaatttttcatcatcaccgacaaaagatggtggattgttggtaattacgactgatgggagattggaacgaattgatagctttggttatttaaaatgggagttaaatttaagaaatattaatgcgttagctactgttgatgaagagcaaaacttagtttatagtgacaatcggattgtcactaaaccattagatgctgtttttattaataatgtgtctaaaaaagtggtagctgaagcgattcaatcacaagaagatccaacgatttattaccttttagcagttagtgatcagatcttaactaacgatcttaaggaccagttacttagttttcaacaattagcaactgatctgacttcacaaggtgtggttttaaaagttaaagagaatcccactcagattagtgatcctgtggggatcattgattatgcaatgatctcaccaactgacttaacacaaaactatcccgctagttggaaaactaacaccttatcattatatggtgataatggtagttttcgtaaagctgatcatccttcatggtttaataatgattctaattttatctcattaccatgattacaatatattactaatttagctaatctttatgaatataaagataacgtttttctttttggtgggaatggtaattgggttaatgcgatcagagtgttcgtcagcaagcaaccaaatacctttctattggggtgtttagaatcaggttttataaagatcttagtactagaccaactgtcacaggttatccttatgcttacttattaagttcattaacttcaaacgatcctgatacccaaaaattattattaggacaaaacaccaactacacccttgttccaaggatagcagttggtggggtaaaacaaggcttaggtgagaataagaatagtgtgttcttatttggtagtatcaccactggcttaaataatacggatgatcctaatcaagtagttttagattcagctaactatacaaacggacagacgaaaaccg

(19) United States
(12) Patent Application Publication
(10) Pub. No.: US 2002/0187162 A1
(43) Pub. Date: Dec. 12, 2002

(54) Use of a live attenuated Mycoplasma gallisepticum strain as a vaccine and vector for the protection of chickens and turkeys from respiratory disease

(76) Inventors: Steven J. Geary, Storrs, CT (US); Lawrence Silbart, Willington, CT (US); Philip Marcus, Storrs, CT (US); Margaret Sekellick, Storrs, CT (US)

Correspondence Address: Cummings & Lockwood, P.O. Box 1960, New Haven, CT, 06506-1960, US

(21) Appl. No.: 10/125,818

(22) Filed: Apr. 19, 2002

Related U.S. Application Data
(60) Provisional application No. 60/285,569, filed on Apr. 21, 2001.

(51) Int. Cl. A61K 39/02; A61K 39/04

(52) U.S. Cl. 424/200.1; 424/248.1

(57) ABSTRACT

The present invention relates to a live cytadherence-deficient M. gallisepticum strain that does not express at least two of three proteins, the Gap-A molecule, crnA protein, and the 45 kDa protein, expressed by wildtype M. gallisepticum Strain R and its use as a vaccine for preventing and protecting birds, especially chickens and turkeys against the respiratory diseases attendant with wildtype Mycoplasma gallispeticum infection. The invention also relates to the use of the vaccine as a vector for the delivery of genes encoding protective antigens from other bacterial and viral avian pathogens, such as avian influenza virus. There is also disclosed a method for identifying the attenuated cytadherence-deficient M. gallisepticum R_{high} or a strain thereof.

### REFERENCE TO RELATED APPLICATION

This application claims benefit under Title 35 U.S.C. § 119(e) of United States Provisional Serial No. 60/285,569 filed Apr. 21, 2001.

### BACKGROUND OF THE INVENTION

1. Field of the Invention

The present invention relates to a live cytadherence-deficient M. gallisepticum strain that does not express at least two of three proteins, Gap-A, crmA, and a 45 kDa protein, found to be expressed by wildtype M. gallisepticum Strain R and its use as a vaccine for preventing the respiratory diseases attendant with wildtype Mycoplasma gallisepticum infection in poultry, such as chickens and turkeys. The vaccine may also be used as a vector for the delivery of genes encoding protective antigens from other bacterial and viral avian pathogens, such as avian influenza virus, and cytokines with antiviral and/or immunomodulatory action, such as avian interferons and interleukins

2. Background of the Invention Viral diseases represent a continuous threat to the poultry industry. Poultry flocks are susceptible to a number of respiratory infections. Some of these infections result in mild illnesses, while outbreaks of others may result in a high number of deaths. Regardless of whether the birds are raised for meat, breeding, or eggs, respiratory infections result in decreased performance and pose disease hazards for other poultry on the premises.

There are numerous causative agents of respiratory infections in birds. Of those causative agents that have been identified over the years, each can be classified as either a virus (e.g., Newcastle disease, infectious bronchitis, laryngotracheitis, quail bronchitis, influenza/parainfluenza), a bacteria (e.g., mycoplasmosis, infectious coryza, endemic fowl cholera, psitticosis/omithosis) or a mold (e.g., aspergillus (brooder pneumonia)).

Among those viral diseases showing high rates of morbidity and/or mortality in poultry is avian influenza (AIV). Signs of the disease depend upon the species affected, age, sex and concurrent infection with other viral and bacterial agents. The signs include sneezing, coughing, rales, lacrimation, huddling, ruffled feathers, edema of the head and face, and sinusitis. The disease is usually accompanied by lower egg production and signs of nervous disorder and diarrhea. When death does not result in the case of meat producing birds, condemnation of the flock may result.

Morbidity and mortality from highly pathogenic strains of AIV can reach 100% in poultry. For example, the “Foreign Animal Disease Report,” USDA 1994 reported that in 1983-1984 an outbreak of a high pathogenicity avian influenza virus resulted in the death of 17 million birds in Pennsylvania, Maryland, New Jersey and Virginia and cost over $500 million (in today's dollars). Over 11 million broiler chicks are produced per week in this region alone, accounting for close to 10% of the U.S. production capacity. Recent outbreaks of the virus in Mexico in 1996 resulted in the death of 18 million chickens when a mild strain mutated to a highly pathogenic strain. Avian influenza is thus an important respiratory virus infecting chickens.

In at least one case, the avian reservoir of AIV led to the generation of influenza outbreaks in humans. This public health hazard was brought to light by the recent emergence of an avian influenza virus in the human population in Hong Kong that resulted in seven human fatalities and the concomitant destruction of approximately 1.3 million birds. A study by the University of Connecticut Cooperative Extension Service estimates the economic loss to the state of Connecticut alone if an outbreak of avian influenza occurred would be in the range of $12 to $166 million depending upon the extent and duration of the outbreak. For every month the industry in Connecticut was affected, around $3.8 million would be lost in feed sales and almost $880 thousand in wages.

Diagnosis of AIV must be based on flock history, symptoms and lesions. Blood tests are often useful in determining whether a flock is infected.

Protection against AIV infection typically entails either eradication and surveillance programs and/or vaccination. Eradication and surveillance for re-occurrence of the disease is effective but very costly. Vaccination against AIV has proven effective. However, immunologic surveillance for the purposes of eradication programs are potentially hindered by the immune response generated by vaccination with whole viruses. Vaccines are nonetheless currently under limited licensure in the U.S. for use in turkeys.

Among those bacterial diseases showing equally high rates of morbidity and/or mortality is Mycoplasma gallisepticum infection. Mycoplasma gallisepticum infection is of considerable economic importance to poultry producers throughout the world. Mycoplasma gallisepticum infection is the major cause of reduced egg production, reduced hatchability, and downgrading of carcasses in the poultry industry today. Mycoplasma gallisepticum infection is widespread and affects many species of birds. Until recently, most chicken flocks and about forty percent of turkey flocks were infected with Mycoplasma gallisepticum.

Chickens and turkeys infected with Mycoplasma gallisepticum display symptoms associated with chronic respiratory diseases/air sac syndrome of chickens and infectious sinusitis of turkeys including air sacculitis when concomitantly infected with E. coli. The condition is triggered by an acute respiratory virus infection such as Newcastle disease or infectious bronchitis. The cause of infectious sinusitis of turkeys is an uncomplicated mycoplasma gallisepticum infection. Birds infected with Mycoplasma gallisepticum evidence respiratory symptoms such as coughing, sneezing and a nasal discharge. In air sacculitis there is extensive involvement of the entire respiratory system and the air sacs are often cloudy and contain a large amount of exudate. There is often a film of exudate covering the liver, as well as the heart muscle and heart sac. Affected birds become droopy, feed consumption decreases and there is a rapid loss of body weight.

Mycoplasma gallisepticum is primarily spread through the egg. Infected hens transmit the organisms and the chick or poult is infected when it hatches. Organisms may also be transmitted by direct contact with infected or carrier birds and possibly by unknown methods. Depending on the system of management, Mycoplasma gallisepticum infection may spread rapidly through an entire flock.

With respect to the broiler chicken industry, losses in the United States alone are estimated at $588 million annually due to Mycoplasma gallisepticum infection. In regard to commercial egg-laying birds, an estimated thirty-seven percent (37%) in the United States are infected with M. gallisepticum resulting in a nearly $132 million annual loss to that industry.

Several approaches are currently employed to reduce the impact of M gallisepticum infection in commercial poultry, including, 1) eradication and surveillance programs, 2) use of antibiotics and antimicrobials, and 3) vaccination.

The primary method for controlling infection has been the eradication of infected birds. Eradication programs have allowed most broiler breeder stock and many commercial egg stocks of poultry to become Mycoplasma gallisepticum-free. Eradication and surveillance programs entail the need for the continuous monitoring of flocks for M. gallisepticum infection, the rapid detection of outbreaks, and the rapid eradication and disposal of infected animals prior to their association with an uncontaminated flock. While eradication and surveillance programs have proven quite beneficial, they have not prevented continuing outbreaks of the disease, due to the difficulty in identifying and isolating all infected animals prior to the association of one or more with a member of an uncontaminated flock.

Antimicrobials, and in particular antibiotics, have been advanced as the answer to M. gallisepticum problem. For example, tilmicosin phosphate has been promoted for the treatment and control of respiratory infections in chicken flocks infected with Mycoplasma gallisepticum. Treatment with antibiotics has resulted in varying success. Antibiotics have also been found to alleviate the signs of M. gallisepticum, but not cure the infection. The upper form of infectious sinusitis usually only can be treated by injecting antibiotics directly into the swollen sinus. High levels of a broad spectrum antibiotic in either feed or drinking water may be used to treat other respiratory conditions. Recently the use of antibiotics in food flocks has come under much criticism.

Vaccination has also been used to control Mycoplasma gallisepticum infectivity. Vaccination includes both (1) routine vaccination of all susceptible poultry, and (2) selective and temporary use of vaccines with the aim of returning the flock to M. gallisepticum-free status.

Chicken interferons have been identified as proteins with the potential to protect chickens and other avian species from viral disease due to their antiviral action (See U.S. Pat. Nos. 5,641,656; 5,885,567; 6,020,465). Some success has been reported against two avian respiratory viruses (J. Interferon Cytokine Res. 19:881-885, 1999. Ibid 21: 1071-1077, 2001). Direct expression of chicken interferon in the respiratory tract as vectored by the vaccine strain of M. gallisepticum described herein may enhance the efficacy of interferon delivery.

In many countries of the world, killed, whole cell M. gallisepticum bacterins are used to effectuate immunity against the disease. Bacterin-based vaccines are claimed to reduce the clinical signs of infection. The commercially-available bacterin vaccines, however, have been found not to prevent colonization of M. gallisepticum . Further, bacterin vaccines appear to protect against infection with the vaccine strain M. gallisepticum from which the bacterins have been obtained, but are reported to be ineffective against many field strains.

In the United States, bacterin-based M. gallisepticum vaccines are no longer sold. Instead the F strain of M. gallisepticum is widely used as a live vaccine. While the F strain is useful in immunizing against M. gallisepticum infectivity, the F strain has moderate virulence to chickens and is highly virulent to turkeys. The virulent nature of this strain is a major disadvantage to its continued use as a live vaccine. Furthermore, the F strain does not result in displacement of virulent field strains of M. gallisepticum.

Two other live vaccines are also available in the United States, identified as ts-11 and 6/85. While these vaccines are less virulent, and less infective than the F strain, they also do not displace virulent field strains. Further neither vaccine affords the same level of protection as the F strain. The ts-11 vaccine does not colonize nor protect turkeys from M. gallisepticum-induced disease.

An effective M. gallisepticum and viral (for example AIV) vaccine must: 1) elicit a protective immune response without inducing adverse reactions for most types of poultry, 2) should not, in the case of live vaccines, revert to virulence, and 3) be readily identifiable as a vaccine strain by easily performed microbiological or serological methods. None of the currently available M. gallisepticum and AIV vaccines meet all of these criteria. Additionally, none of these vaccines is suitable, safe, or protective for use in the turkey industry.

While the vaccine described and associated vaccination techniques are generally referred to herein as intended for chickens and turkeys, the invention is not limited to these but includes ostrich, emus, ducks, geese, quails and exotic birds such as parrots, cockatoos, cockatiels and other commercially valuable birds.

### SUMMARY OF THE INVENTION

This invention relates to the use of a live cytadherence-deficient M. gallisepticum strain. As a vaccine for preventing the respiratory diseases attendant with wildtype Mycoplasma gallisepticum infection in poultry, such as chickens and turkeys. This strain does not express at least two of three proteins, Gap-A, crnA, and a 45 kDa protein, found to be expressed by wildtype M. gallisepticum Strain R. The invention also relates to the use of a such a live cytadherence-deficient M. gallisepticum strain to carry additional heterologous genes for expressing an antigen of a poultry infectious virus, another bacterial pathogen or a cytokine with antiviral or adjuvant properties, such as interferons.

As disclosed in an article by Papazisi et al. entitled “Analysis of Cytadherence-Deficient, GapA-Negative Mycoplasma gallisepticum Strain R,” 68 Infection and Immunity 6643-6649, 2000 which constitutes part of this disclosure and is incorporated in its entirety herein by reference thereto, it has previously been discovered by one of the inventors of the present invention, that a high passage (about 164 passages) M. gallisepticum strain R (designated Rhigh) has exceedingly lower virulence when compared to wild-type M. gallisepticum strain R cultured with a low number of passages (R_{low}). Attenuation of the Rhigh strain has been determined to be related to significantly lower cytadhesion to respiratory tissue in poultry, and a lower ability of the R_{high} strain to colonize in vivo as compared to the R_{low} strain. As shown in the Papazisi et al. article, DNA analysis of R_{high} versus R_{low} strains demonstrates that the R_{high} strain does not express genes encoding for three proteins: (1) the cytadhesin molecule GapA; (2) p116 expressed from a gene designated crmA that is located immediately downstream of gapA (previously referred to as ORF 2) in the operon, such protein having about 41% overall amino acid identity with M. pneumoniae ORF6 and MgpC of Mycoplasma genitalium; and (3) p45 encoded by a sequence outside of the gapA operon. The lack of expression of GapA in R_{high} appears to be due to a genetic alteration such that a stop codon is formed resulting in premature termination of translation. The Papazisi et al. reference suggests that p116 and/or p45 may play a role either in the presence and/or localization of Gap A or the functional capacity of some other cytadherence-related molecule.

It has been found that the R_{high} strain of M. gallisepticum described above may be used to elicit a high degree of immunity to infection in poultry to the wild-type (R_{low}) strain of M. gallisepticum. Such finding is particularly important, in that the differences in DNA sequence and protein expression between R_{low} and R_{high} strains can be used to differentiate between an immunized bird, and one infected with the wild-type mycoplasma. That is, immunization with the R_{high} strain does not interfere with detection of infection with the R_{low} strain. For example, birds exposed to the R_{high} strain will not develop anti-GapA antibodies while birds exposed to the R_{low} strain will develop antibodies to GapA. Likewise, birds exposed to one strain will demonstrate a distinctly different random amplified polymorphic DNA profile than birds exposed to the other strain.

It has been found by the inventors herein that the R_{high} strain of M. gallisepticum transformed with wild-type gapA (hereinafter, “modified-R_{high} strain”) still remains cytadherence-deficient and pathogenic. Surprisingly, the modified-R_{high} strain of M. gallisepticum is useful for stimulating antibody immune response against the infective wild-type M. gallisepticum R strain, and may provide enhanced immunity in some avian species greater than provided by the R_{high} strain of M. gallisepticum.

It is known that a recombinant fowlpox virus expressing the H5 hemagglutinin of avian influenza virus (AIV) induces nearly 100% protection from a highly pathogenic challenge of AIV. A major advantage of vaccinating against AIV with a vaccine containing H5 hemagglutinin is that it permits vaccination with a single protective AIV antigen (HA) which would not interfere with immunologic surveillance for infection with wild-type AIV and therefore will not interfere with the implementation of an eradicator program. Instead it should be viewed as a tool to facilitate assessment of any AIV eradication program.

It also has been found that by transforming the R_{high} strain, and modified-R_{high} strain, to incorporate a heterologous gene for an antigen of an infectious agent such that there is co-expression of antigens from the R_{high} strain and the infectious agent, a vaccine is produced that permits immunity to be gained by the animal to both wild-type R_{low} M. gallisepticum and to the other infectious agent. Thus, any number of bacterial and viral antigens derived from avian respiratory or other pathogens may be delivered by the R_{high} and modified-R_{high} M. gallisepticum vector. In addition, delivery and expression of avian cytokine genes, such as interferons, also should be possible.

In particular, by immunizing a bird with a R_{high} or and modified-R_{high} M. gallisepticum transformed with AIV H5 hemagglutinin one may produce a bird that is protected both against wild-type M. gallisepticum R-strain infection and AIV infection. The capacity of the M. gallisepticum/AIV HA construct (“GTHA”) to protect birds from challenge with virulent M. gallisepticum and to induce a serological response to the AIV H5 constitutes a marked advance sufficiently compelling to warrant providing the attenuated strain of M. gallisepticum as both a modified live vaccine and as an H5 expression vector.

### DETAILED DESCRIPTION OF THE INVENTION

As detailed above, vaccination of poultry against M. gallisepticum infectivity in the past has entailed either vaccination with bacterins, or use of the F-strain, ts-11 or 6/85 strains of the M. gallisepticum bacterium as a live vaccine. Bacterin-based vaccinations have not been found to be very effective.

While the present invention is not limited in any manner by the following hypothesis, the inventors herein hypothesize that the failure of bacterin-based vaccines to protect vaccinated birds from colonization may be explained by the ability of M. gallisepticum strains to change their antigenic signature by switching the antigens expressed on the cell surface (phenotypic variations) in response to the selective pressure of antibody, thereby providing the organisms with a means of evading the host immune response generated to the antigen of the original bacterin.

Furthermore, vaccination with the ts-11 and 6/85 strains has not been found to provide the degree of protection against infectious M. gallisepticum that is required in the field. While the F strain of M. gallisepticum has been found to provide significantly more protection, that strain also is associated with moderate virulence in chickens resulting in a decrease in egg laying over a period of time and a high degree of virulence in turkeys.

The inventors herein have now found that a high level of protection against virulent strains of M. gallisepticum can be obtained by inoculation with a live cytadherence-deficient M. gallisepticum strain that does not express at least two of three proteins, Gap-A, crmA protein and a 45 kDa protein. In particular, a strain of M. gallisepticum, designated R_{high} has been identified which fails to express the three proteins normally expressed in wild-type infectious strains of M. gallisepticum. All three of these proteins, namely, Gap-A, p116 or CrmA, and p45, which appear to be involved in the cytadherence competence of the bacterium with respect to avian respiratory tissue, and are characterized in the Papazisi et al. article entitled “Analysis of Cytadherence-Deficient, GapA-Negative Mycoplasma gallisepticum Strain R,” Infection and Immunity 68:6643 -6649, 2000.

The inventors have also discovered that live cytadherence-deficient M. gallisepticum strains in particular those like the R_{high} that do not express at least two of three proteins, Gap-A, crmA, and a 45 kDa protein, found to be expressed by wildtype M. gallisepticum Strain R, can be used as a vector of other bacterial and viral antigens derived from avian respiratory pathogens, so as to produce a vaccine capable of protecting not only against M. gallisepticum infectivity, but also, against infectivity by the other avian respiratory pathogens.

In accordance with an embodiment of the present invention, there is provided a vaccine comprising the R_{high} strain or modified-R_{high} strain of M. gallisepticum transformed with a heterologous gene for H5 hemagglutinin of avian influenza virus. Such vaccine may be used to protect birds from infection with both virulent strains of M. gallisepticum and avian influenza virus.

In another embodiment, the ability of the vaccine to stimulate or increase the antibody immune response against infections M. gallisepticum is exploited.

The invention will be further illustrated by the following exemplification:

Example 1: Transformation of R_{high} strain of M. gallisepticum with H5HA

Polymerase Chain Reaction (PCR) Protocol

PCR reactions were carried out in a total volume of 50 μl containing 50 ng of template, 250 mM each of dATP, dTTP, dGTP, and dCTP, 1.5 MM MgCl₂, 400 ng of each primer (synthesized by the University of Connecticut Biotechnology Center) and 2.5 units AmpliTaq (Applied Biosystem/Perkin Elmer, Norwalk, Conn.). The targeted regions were amplified according to the following conditions: 25 cycles of 94° C. for 1 minute, 5° C. below the melting temperature (Tm) of the primer for 1 minute, 72° C. for 2 minutes followed by one cycle at 72° C. for 10 minutes.

DNA Sequencing Protocol

Sequencing was performed by primer walking at the Keck Foundation Biotechnology Resource Laboratory at Yale University-School of Medicine in New Haven, Conn. and at the University of Connecticut Macromolecular Characterization Facility of the Biotechnology Center. DNA sequencing reactions were performed using a Taq DyeDeoxy Terminator Cycle Sequencing Kit according to the protocol provided with the Perkin-Elmer kit and analyzed on an Applied Biosystems 373A Stretch DNA Sequencer. DNAMAN (Lynnon BioSoft, Quebec, Canada) was used for the alignments of the amino acids. The stem-loop structures were determined using DNAMAN and MacDNASIS (Hitachi Software Engineering America, San Bruno, Calif.)

Construction of H5HA Transposon

The Avian Influenza Virus Hemagglutinin (H5HA) gene from a cDNA clone was amplified by PCR utilizing primers incorporating BamH1 sites and designed to eliminate the first 48 nucleotides (16 amino acids) of the N-terminal sequence which codes for the signal peptide. The signal peptide was eliminated from this construct to allow for the translation and storage of H5HA within the cytosol thereby preventing possible presentation of viral hemagglutinin on the surface of the mycoplasma, thereby reducing the risk of possible increased cytadherence due to HA expression on the surface of R. PCR amplified H5HA was digested with BamH1 and ligated into BamH1 digested pISM 2062. Products of the ligation, Plasmid, pISM 2062, containing the modified Transposon Tn4001mod, were used as the vector to insert H5HA into a clonal isolate of R_{high}.

Transformation of M. gallisepticum R_{high} With Tn4001-AIV H5HA

M. gallisepticum R_{high} was transformed with Tn4001-AIV H5HA by a modified method of King and Dybvig (King, K. W. and K. Dybvig, Plasmid Transformation of Mycoplasma mycoides subspecies mycoides is promoted by high concentrations of ethylene glycol, Plasmid 26: 108-115 (1991)). Organisms from 1 ml overnight culture were harvested and washed in ST buffer (500 MM sucrose; 10 mM Tris, pH 6.5). Washed cells were suspended in 500 μl 100 MM CaCl₂ and incubated on ice for 30 minutes. Yeast transfer RNA (20 μg) and the Tn4001 vector DNA (10 μg) were added along with 4 ml 40% polyethylene glycol (PEG), and the resulting suspension was incubated for 2 minutes at room temperature. Thereafter the suspension was diluted with 20 ml ST buffer and the cells were harvested by centrifuigation at 10,000×g for 15 minutes. The cell pellet was suspended in 2 ml Frey's medium and incubated at 37° C. for 3 hours. After the incubation was completed, 50 μg gentamicin ml⁻¹ was added, and the broth cultures were incubated at 37° C. overnight, then plated on solid medium containing 15 μg gentamicin ml⁻¹ and further incubated at 37° C. Clones were picked and checked for the presence of both H5HA and gentamicin resistant genes by PCR analyses using specific primers. A single clone of M. gallisepticum R_{high} transformed with Tn4001-AIV H5HA designated as GTHA was selected for further analyses. The DNA sequence was determined as described below (using oligonucleotide primers synthesized from the ends of the IS element and primer walking).

Example 2: Preparation of Modified-R_{high} strain of M. gallisepticum

Polymerase Chain Reaction DNA Sequencing Protocols

PCR reactions and DNA sequencing were performed as described in Example 1.

Construction of Modified-R_{high} Strain of M. Gallisepticum

Polymerase chain reaction products were cloned into the PCRII vector of the TA cloning kit according to the manufacturers' protocol (Invitrogen). The vectors containing the correct inserts were transformed into E. coli INVαF′ (Invitrogen) competent cells according to the manufacturer's protocol. White colonies were selected and the inserts were sequenced, as described below.

Plasmid, pISM2062, containing the modified Transposon Tn4001mod (15), was used as the vector to insert wild-type gapA into a GapA⁻, clonal isolate of R_{high}. A 4112 bp fragment, containing the gapA gene, was amplified from M. gallisepticum strain R_{low} using forward (5′ gggggatccagaccaaacttccctaac 3′) and reverse (5′ gggggatccagcaaaatcatcacttag 3′) primers. Tn4001mod contains a unique BamH1 site at the end of the IS256L arm. The fragment containing the gapA gene was cloned into the BamH1 site of the Tn4001mod. Recombinant clones were selected with the insert oriented so that gapA was transcribed from an outward-reading promoter in IS256L.

R_{high} was transformed with the gapA gene by a modified method of King and Dybvig. Organisms from 1 ml overnight culture were harvested and washed in ST buffer (500 mM sucrose; 10 mM Tris, pH 6.5). The washed cells were suspended in 500 μl 100 mM CaCl₂ and incubated on ice for 30 minutes. Yeast transfer RNA (20 μg) and the Tn4001-gapA vector DNA (10 μg) were added along with 4 ml 40% polyethylene glycol (PEG), and the suspension was incubated for 2 minutes at room temperature. The suspension was diluted with 20 ml ST buffer and the cells were harvested by centrifugation at 10,000×g for 15 minutes. The cell pellet was suspended in 2 ml Frey's medium and incubated at 37° C. for 3 hours.

Following incubation, 50 ug gentamicin ml⁻¹ were added, and the broth cultures were incubated at 37° C. overnight then plated on solid medium containing 15 mg gentamicin ml⁻¹ and incubated further at 37° C. Single colonies were picked, propagated and analyzed by immunoblot, using anti-GapA serum, for the expression of gapA and by Southern hybridization of HindIII digested genomic DNA using both 32P labeled gapA as a probe and then reprobed with ³²p labeled Tn4001 DNA. The Southern hybridization conditions as used follow: Probes were incubated with the blot (42° C. with 45% (v/v) formamide) for 16 hours. The filters were washed twice with 2X SSC (300 mM NaCl,30 mM sodium citrate, pH 7.0)-0.1% SDS and 0.2X SSC-0.1% SDS for 3 minutes at room temperature followed by two additional washes at 5° C. in 0.16X SSC-0.1% SDS for 15 minutes. The filters were dried and exposed to film (Fuji Rx Film, Fisher Scientific, Pittsburgh, Pa.) using intensifying screens. Those clones which were positive for GapA by immunoblot and possessed the complemented wild-type copy of the gapA were analyzed further. The restriction fragment containing the Tn4001-gapA insert was excised and cloned into pBluescript SK11+(Stratagene) which had been previously digested with HindIII, and treated with calf intestinal alkaline phosphatase, at a 2:1 insert DNA to vector ratio. The ligation mixture was transformed into E. coli XL1-Blue competent cells and selected for on LB plates containing 100 μg ampicillin ml⁻¹ and 25 μg X-Gal ml⁻¹. The DNA sequence was determined as described above using oligonucleotide primers synthesized from the ends of the IS element and primer walking.

The vaccines are used for preventing the viral infections of fowl as above set forth in the conventional manner, namely by administering an effective amount of the vaccine to the fowl. For example, the vaccine can be administered in ovo to a chick or an adult fowl. The treatment can in the case of administration to a chick or adult fowl, can be for example by oral, mucosal or aerosol administration, by injection or via drinking water. Aerosol administration is preferred for mass immunization.

In the same manner the vaccines can be used to immunize susceptible fowl, for example chickens and turkeys by delivering an immunologically effective amount of the vaccine.

The vaccine compositions can be formulated in the conventional manner with or without carriers or adjuvants. If carriers or adjuvants are used the conventional carriers and adjuvants should be employed, and particularly those used in formulating live vaccines.

A series of experiments were carried out in order to evaluate the protection afforded by vaccines in accordance with the invention and to determine the pathogenicity of the vaccines and thereby approximate dosage amounts. The Tables which follow are directed to the results of these experiments.

TABLE 1
Pathogenicity of M. gallisepticum R_{low} and R_{high} strains for young chickens

| | | | | Tracheal lesion | |
|---|---|---|---|---|---|
| | | | Necropsy | scores | Tracheal |
| Group | Vaccine | Challenge | (post-challenge) | (mean ± sem)* | isolates* |
| 1 | — | Medium | 2 weeks | 0.40 ± 0.20^{a} | 0/5^{a} |
| 2 | — | 5 × 10⁷ R_{high} | 2 weeks | 0.60 ± 0.20^{a} | 0/5^{a} |
| 3 | — | 1 × 10⁸ R_{high} | 2 weeks | 0.80 ± 0.24^{a} | 0/5^{a} |
| 4 | — | 5 × 10⁷ R_{low} | 2 weeks | 2.20 ± 0.68^{b} | 5/5^{b} |
| 5 | — | 1 × 10⁸ R_{low} | 2 weeks | 2.40 ± 0.37^{b} | 5/5^{b} |

*Means of the same column with the same letter are not significantly different (p < 0.05).

TABLE 2
Comparison of the pathogenicity of different challenge doses of M. gallisepticum R_{low}
strain vs. GT5 for young chickens

| | | | | Tracheal lesion | |
|---|---|---|---|---|---|
| | | | Necropsy | scores | Tracheal |
| Group | Vaccine | Challenge | (post-challenge) | (mean ± sem)* | isolates* |
| 1 | — | 1 × 10⁷ R_{low} | 2 weeks | 1.94 ± 1.01^{a} | 7/8^{a} |
| 2 | — | 2 × 10⁶ R_{low} | 2 weeks | 1.36 ± 0.99^{a} | 5/7^{a} |
| 3 | — | 4 × 10⁵ R_{low} | 2 weeks | 1.19 ± 1.03^{a} | 6/8^{a} |
| 4 | — | 3 × 10⁸ GT5 | 2 weeks | 0.13 ± 0.22^{b} | 0/8^{b} |
| 5 | — | Medium | 2 weeks | 0.25 ± 0.25^{b} | 0/4^{b} |

*Means of the same column with the same letter are not significantly different (p < 0.05).

TABLE 3
Evaluation of GT5 protection against virulent M. gallisepticum R_{low} in young chickens

| | | | | Tracheal lesion | |
|---|---|---|---|---|---|
| | | | Necropsy | scores | Tracheal |
| Group | Vaccine | Challenge | (post-challenge) | (mean ± sem)* | isolates* |
| 1 | 3 × 10⁸ | 1 × 10⁷ R_{low} | 2 weeks | 0.43 ± 0.17^{de} | 1/7^{a} |
| | GT5 | | | | |
| 3 | Medium | 1 × 10⁷ R_{low} | 2 weeks | 1.75 ± 0.56^{a} | 6/6^{b} |
| 5 | PBS | 1 × 10⁷ R_{low} | 2 weeks | 1.33 ± 0.37^{ab} | 5/6^{b} |
| 7 | PBS | PBS | 2 weeks | 0.25 ± 0.25^{e} | 0/6^{a} |
| 2 | 3 × 10⁸ | 1 × 10⁷ R_{low} | 4 weeks | 0.79 ± 0.36^{cd} | 0/7^{a} |
| | GT5 | | | | |
| 4 | Medium | 1 × 10⁷ R_{low} | 4 weeks | 1.08 ± 0.34^{bc} | 2/6^{b} |
| 6 | PBS | 1 × 10⁷ R_{low} | 4 weeks | 1.50 ± 0.50^{ab} | 4/6^{b} |
| 8 | PBS | PBS | 4 weeks | 0.40 ± 0.20^{e} | 0/6^{a} |

*Means of the same column with the same letter are not significantly different (p < 0.05).

While the invention has been described with respect to preferred embodiments, those skilled in the art will readily appreciate that various changes and/or modifications can be made to the invention without departing from the spirit or scope of the invention as defined by the appended claims. All documents cited herein are incorporated in their entirety herein.

What is claimed is:

1. A method of avian vaccination against virulent strains of Mycoplasma gallisepticum comprising the step of administering to a bird an immunogenically effective amount of an immunogen comprising a cytadherence-deficient Mycoplasma gallisepticum bacterium characterized by its inability to express at least two of three proteins expressed by wild-type Mycoplasma gallisepticum R in the group consisting of: the cytadhesin molecule GapA, the crnA protein, and the 45 kDa protein.

2. The method of claim 1 wherein the cytadherence-deficient Mycoplasma gallisepticum bacterium is Mycoplasma gallisepticum R_{high}.

3. The method of claim 1 wherein the cytadherence-deficient Mycoplasma gallisepticum bacterium is characterized by its inability to express the 45 kDa protein expressed by wild-type Mycoplasma gallisepticum R.

4. A method of avian vaccination against virulent strains of Mycoplasma gallisepticum comprising the step of administering to a bird an immunogenically effective amount of an immunogen comprising a cytadherence-deficient Mycoplasma gallisepticum bacterium characterized by its inability to express the cytadhesin molecule GapA and the crmA protein both of which are expressed by wild-type Mycoplasma gallisepticum R, and having been artificially transformed with a gene encoding an antigen from a heterologous bacterial and/or viral avian pathogen.

5. A method of avian vaccination against virulent strains of Mycoplasma gallisepticum comprising the step of administering to a bird an immunogenically effective amount of an immunogen comprising a cytadherence-deficient Mycoplasma gallisepticum bacterium characterized by its inability to express the cytadhesin molecule GapA and the crmA protein expressed by wild-type Mycoplasma gallisepticum R, and having been artificially transformed with a gene encoding AIV H5.

6. A vaccine composition for inducing or increasing the antibody immune response against infectious Mycoplasma gallisepticum said vaccine comprising a cytadherence-deficient Mycoplasma gallisepticum bacterium characterized by its inability to express the cytadhesin molecule GapA and the crmA protein both of which are expressed by wild-type Mycoplasma gallisepticum R.

7. A vaccine composition for inducing or increasing the antibody immune response against infectious Mycoplasma gallisepticum comprising a cytadherence-deficient Mycoplasma gallisepticum bacterium characterized by its inability to express the cytadhesin molecule GapA and the crmA protein both of which are expressed by wild-type Mycoplasma gallisepticum R, and having been artificially transformed with a gene encoding an antigen from a heterologous bacterial and/or viral avian pathogen.

8. A vaccine composition for inducing or increasing the antibody immune response against infectious Mycoplasma gallisepticum comprising a cytadherence-deficient Mycoplasma gallisepticum bacterium characterized by its inability to express the cytadhesin molecule GapA and the crmA protein both of which are expressed by wild-type Mycoplasma gallisepticum R, and having been artificially transformed with a gene encoding AIV H5HA.

9. A cytadherence-deficient Mycoplasma gallisepticum bacterium characterized by the inability to express the cytadhesin molecule GapA and the crmA protein both of which are expressed by wild-type Mycoplasma gallisepticum R used as a vaccine against infectious Mycoplasma gallisepticum.

10. The cytadherence-deficient Mycoplasma gallisepticum bacterium of claim 9 further characterized in expressing the H5 hemagglutinin of the avian influenza virus.

11. A method for inducing an immunological response in a bird comprising administering to the bird a cytadherence-deficient Mycoplasma gallisepticum bacterium according to claims 9.

12. The method of claim 11 wherein the route of administration is selected from the group consisting of parenteral administration, mucosal administration, aerosol administration, oral administration, transdermal administration and in ovo administration.

13. A method for identifying the attenuated cytadherence-deficient Mycoplasma gallisepticum R_{high} or a strain derived therefrom, which functions as an effective component of an attenuated Mycoplasma gallisepticum vaccine in avian species, comprising the step of determining that a Mycoplasma gallisepticum infected bird does not exhibit anti-GapA antibodies.

14. A method for identifying the attenuated cytadherence-deficient Mycoplasma gallisepticum R_{high} or a strain derived therefrom, which functions as an effective component of an attenuated Mycoplasma gallisepticum vaccine in avian species, comprising the step of determining that Mycoplasma gallisepticum obtained from a Mycoplasma gallisepticum-infected bird strain does not express GapA and crmA.

15. The method of claim 14 further comprising the step of determining that the Mycoplasma gallisepticum does not express the 45 k-Da protein found in wild-type Mycoplasma gallisepticum strain R.

16. A method for preventing viral infection in birds by the Mycoplasma gallisepticum virus comprising administering an effective amount of a vaccine comprising a cytadherence-deficient Mycoplasma gallisepticum bacterium characterized by its inability to express at least two of the three proteins expressed by the wild-type Mycoplasma gallisepticum R: the cytaadhesion molecule GapA, the crmA protein and the 45 kDA protein.

17. Method according to claim 16 wherein said vaccine is administered orally.

18. Method according to claim 16 whiere said vaccine is administered in ovo.

19. A method according to claim 6 wherein said vaccine composition includes a carrier.

20. A method for the preparation of a live vaccine effective against virulent strains of Mycoplasma gallisepticum comprising passaging virus in a culture on a suitable medium for a sufficient number of times to reduce its pathogenicity while retaining its immunogenicity, harvesting the attenuated virus and further processing the harvested material to produce a vaccine comprising a cytadherence-deficient Mycoplasma gallisepticum bacterium characterized by its inability to express at least two of the three proteins expressed by the wild-type Mycoplasma gallisepticum R: the cytaadhesion molecule GapA, the crmA protein and the 45 kDA protein.

21. A live vaccine effective against virulent strains of Mycoplasma gallisepticum according to claim 6 encoded by a nucleic acid molecule having the nucleic acid sequence according to SEQ ID NO. 1.

22. A live vaccine for producing antiviral and immunomodulatory effects in chickens comprising a vaccine according to claim 6 having incorporated therein at least one immunostimulator selected from the group consisting of chicken interferons, cytokines and chemokines, wherein the Mycoplasma gallisepticum serves as a vector.

23. A live vaccine effective against virulent strains of Mycoplasma gallisepticum containing heterologous genes for expressing an antigen of a member selected from the group consisting of a poultry infections, viral and bacterial pathogens and cytokines.
